(19) United States
(12) Patent Application Publication
(10) Pub. No.: US 2013/0203615 A1
(43) Pub. Date: Aug. 8, 2013

(54) ANTIANDROGEN THERAPY MONITORING METHODS AND COMPOSITIONS

(71) Applicants: John Carl GROSKOPF, San Diego, CA (US); Harry George RITTENHOUSE, Los Osos, CA (US)

(72) Inventors: John Carl GROSKOPF, San Diego, CA (US); Harry George RITTENHOUSE, Los Osos, CA (US)

(73) Assignee: GEN-PROBE INCORPORATED, San Diego, CA (US)

(21) Appl. No.: 13/567,754

(22) Filed: Aug. 6, 2012

Related U.S. Application Data
(60) Provisional application No. 61/515,732, filed on Aug. 5, 2011.

(51) Int. Cl. C12Q 1/68; G01N 33/68

(52) U.S. Cl. CPC C12Q 1/6886; G01N 33/6893
USPC 506/9; 435/6.13; 435/6.11; 435/6.12; 435/7.4; 435/7.92; 506/16

(57) ABSTRACT

The present invention provides methods of evaluating the effectiveness of an antiandrogen therapy in a human by comparing the pre- and post antiandrogen treatment levels of an androgen modulated diagnostic marker and a prostate-specific, androgen independent, diagnostic marker in the human. Methods utilizing these markers are also provided that are useful for identifying antiandrogen compounds capable of killing prostate cancer cells, and identifying a human suspected of responding more, or less, favorably to treatment with an antiandrogen compound and monitoring the treatment thereof. Kits and compositions related to these methods are also provided.

### CROSS-REFERENCE TO RELATED APPLICATIONS

This application claims priority to U.S. Patent Application No. 61/515,732, filed Aug. 5, 2011, and is incorporated herein by reference.

### FIELD OF THE INVENTION

The present invention relates to compositions and methods for monitoring cancer therapy, including but not limited to, cancer markers.

### BACKGROUND OF THE INVENTION

Prostate cancer is the second most frequently diagnosed cancer and the sixth leading cause of cancer death in males, accounting for 14% (903,500) of the total new cancer cases and 6% (258,400) of the total cancer deaths in males in 2008. Though incidence rates have been found to vary by over 25% from country to country, the highest recorded rates are primarily in developed countries, particularly in Australia, New Zealand, Europe, and North America. The increased incidence rates in developed countries can be largely attributed to increased patient screening.

Androgens play an important role in the development, growth, and progression of prostate cancer. The two most important androgens in this regard are testosterone and dihydrotestosterone. The testes synthesize about 90% of testosterone and the remainder is synthesized by the adrenal glands. Testosterone is further converted to the more potent androgen dihydrotestosterone by the enzyme steroid 5α-reductase, which is localized primarily in the prostate. In the testes and adrenal glands, the last step in the biosynthesis of testosterone involves two reactions that act in sequence and are both catalyzed by a single enzyme, the cytochrome P450 monooxygenase 17α-hydroxylase/17,20-lyase enzyme (CYP17).

Androgen deprivation was introduced as as therapy for advanced and metastatic prostate cancer in 1941. Thereafter, androgen ablation therapy has been shown to produce the most beneficial responses in multiple settings in prostate cancer patients. Orchiectomy (either surgical or medical with a GnRH agonist) remains the standard treatment option for most prostate cancer patients. Medical and surgical orchiectomy reduces or eliminates androgen production by the testes but does not affect androgen synthesis in the adrenal glands. It has been found that combination of orchiectomy together with antiandrogen therapy to inhibit the action of adrenal androgens significantly prolongs the survival of prostate cancer patients. It has also been demonstrated that testosterone and dihydrotestosterone occur in recurrent PCA tissues at levels sufficient to activate androgen receptors. In addition, using microarray-based profiling of isogenic PCA xenograft models, it was found that a modest increase in androgen receptor mRNA was the only change consistently associated with the development of resistance to antiandrogen therapy. As such, potent and specific compounds that inhibit androgen synthesis in the testes, adrenals, and other tissue may be more effective for the treatment of prostate cancer.

Screening for prostate cancer and determining the effectiveness of prostate cancer treatments, until relatively recently, involved both palpation of the prostate by digital rectal examination (DRE) and assay of plasma or serum levels of prostate specific antigen (PSA). Serum PSA measurements were regarded as the best conventional serum marker available to detect prostate cancer and whether it had been successfully targeted by a therapeutic. However, it has become well-known that despite its sensitivity, the use of PSA is limited by a significant lack of specificity. As such, evaluating PSA levels has resulted in the performance of a substantial number of unnecessary prostatic biopsies in patients on the front end, and also provides an indirect and non-specific indication of therapeutic efficacy after treatment.

If prostate cancer spreads beyond the prostate it has a strong tendency to metastasize to bone. A bone scan is one of the most commonly used tests to determine if this progression has occurred and whether treatment has been successful. The procedure involves injection of a radioactive tracer into a patient and waiting for it to distribute throughout the skeleton. This type of test is used to detect areas of bone damage due to cancer, infection, or other causes since it can detect new areas of bone growth or breakdown throughout the skeleton. However, a bone scan does not distinguish between normal and abnormal bone growth by itself. For example, the tracer used in the scan may accumulate in certain areas of the bone, indicating one or more hot spots. Hot spots may be caused by a number of things, including a fracture that is healing, bone cancer, a bone infection (osteomyelitis), arthritis, or a disease of abnormal bone metabolism (such as Paget's disease). In contrast, certain areas of the bone may lack the presence of tracer, indicating one or more cold spots. Cold spots may be caused by a certain type of cancer (such as multiple myeloma) or lack of blood supply to the bone (bone infarction).

When it comes to monitoring drug response in prostate cancer patients, particularly those with advanced disease, bone scans, CAT scans, serum PSA levels, and/or circulating tumor cell levels are utilized in the clinic to evaluate therapy effectiveness. These assays, however, are not particularly informative due to their limitations. The limitations of bone scans are noted above—although they may be direct they lack specificity. Serum PSA level, on the other hand, provides only an indirect measurement and is subject to circulating androgen levels. Circulating tumor cell measurements are also limited by the low sensitivity of the technology.

Accordingly there is a need in the art for a non-invasive direct and rapid method of measuring the efficacy of hormonal and chemotherapy prostate cancer treatments. There is also a need in the art to determine optimal dosing of prostate cancer therapeutics in patients to maximize therapeutic benefit, while decreasing the risk of adverse dosing-related events. The present invention addresses these needs and others in the art.

The present description refers to a number of documents, the content of which is herein incorporated by reference with regard to each issue for which they are cited, and related issues.

### SUMMARY OF THE INVENTION

In one embodiment a method of evaluating the effectiveness of an antiandrogen treatment in a cancer patient is provided, comprising: (a) contacting a sample obtained from the patient with an androgen modulated diagnostic marker detection reagent and a prostate-specific, androgen independent, diagnostic marker detection reagent and detecting, if present, the androgen modulated diagnostic marker and the prostate-specific, androgen independent, diagnostic marker in the sample; (b) comparing the pre- and post antiandrogen treatment levels of the androgen modulated diagnostic marker in a patient; and c. comparing the pre- and post-antiandrogen treatment levels of the prostate-specific, androgen independent, diagnostic marker in a patient; wherein detecting a statistically significant pre-/post-treatment decrease in the androgen modulated diagnostic marker, and a statistically significant pre-/post-treatment increase in the prostate-specific, androgen independent, diagnostic marker in the patient indicates therapeutic effectiveness of the antiandrogen treatment.

In another embodiment, a method of identifying an antiandrogen compound capable of killing prostate cancer cells is provided, comprising: (a) contacting a sample obtained from a human with an androgen modulated diagnostic marker detection reagent and a prostate-specific, androgen independent, diagnostic marker detection reagent and detecting, if present, the androgen modulated diagnostic marker and the prostate-specific, androgen independent, diagnostic marker in the sample; (b) comparing the pre- and post-antiandrogen treatment levels of an androgen modulated diagnostic marker in the human, wherein the human received treatment with an antiandrogen compound or a suspected antiandrogen compound; and (c) comparing the pre- and post-antiandrogen treatment levels of a prostate-specific, androgen independent, diagnostic marker in the human; wherein detecting a statistically significant pre-/post-treatment decrease in the androgen modulated diagnostic marker, and a statistically significant pre-/post-treatment increase in the prostate-specific, androgen independent, diagnostic marker in the human identifies an antiandrogen compound capable of killing prostate cancer cells in a human.

Methods of identifying a human suspected of responding more, or less, favorably to treatment with an antiandrogen compound and monitoring the treatment thereof are also provided, comprising: (a) determining whether a human is suspected of responding more, or less, favorably to treatment with a first antiandrogen compound by examining a sample obtained from the patient for the presence, or absence, of a first androgen regulated marker, wherein detection of the presence of the first marker indicates that the human is suspected of responding more, or less, favorably to treatment with the first antiandrogen compound; (b) for a human identified as suspected of responding more favorably to treatment with the first antiandrogen compound: (1) examining a sample obtained from the human by contacting the sample with an androgen modulated diagnostic marker detection reagent and a prostate-specific, androgen independent, diagnostic marker detection reagent and detecting, if present, one or more androgen modulated diagnostic markers and a prostate-specific, androgen independent, diagnostic marker in the sample; and (2) comparing the pre- and post-treatment levels of the one or more androgen modulated diagnostic markers in the human, and comparing the pre- and post-treatment levels of the prostate-specific, androgen independent, diagnostic marker(s) in the human, wherein the human received treatment with the particular antiandrogen compound; and (c) for a human identified as suspected of responding less favorably to treatment with the first antiandrogen compound: (1) examining a sample obtained from the human by contacting the sample with an androgen modulated diagnostic marker detection reagent and a prostate-specific, androgen independent, diagnostic marker detection reagent and detecting, if present, one or more androgen modulated diagnostic markers and a prostate-specific, androgen independent, diagnostic marker in the sample; and (2) comparing the pre- and post-treatment levels of the one or more androgen modulated diagnostic markers in the human, and comparing the pre- and post-treatment levels of the prostate-specific, androgen independent, diagnostic marker(s) in the human, wherein the human received treatment with a second antiandrogen compound; wherein detecting a statistically significant pre-/post-treatment decrease in the androgen modulated diagnostic marker, and a statistically significant pre-/post-treatment increase in the prostate-specific, androgen independent, diagnostic marker in the human identifies a therapeutic benefit of the first or second antiandrogen compound in the respective human that received the first or second antiandrogen compound. In a related embodiment, the marker identified in the above method is not an androgen regulated marker, but if present in a human suggests that the human would respond favorably to treatment with the first antiandrogen compound. The present invention contemplates identification and monitoring the treatment of these humans in accordance with the methods described herein.

Methods of evaluating the effectiveness of an antiandrogen treatment in a subject are also provided, comprising: (a) exposing a sample comprising the AMDM to a primer that is complementary to the AMDM and a polymerase under conditions such that an extension product of the primer is generated, and exposing a sample comprising the PSAIDM to a primer that is complementary to the PSAIDM and a polymerase under conditions such that an extension product of the primer is generated; (b) detecting the extension product of the primer that is complementary to the AMDM, and detecting the extension product of the primer that is complementary to the PSAIDM; and (c) comparing a pre- and post-antiandrogen treatment level of the AMDM in the subject; and comparing a pre- and post-antiandrogen treatment level of the PSAIDM in the subject, wherein detecting a statistically significant pre-/post-antiandrogen treatment decrease in the AMDM, and a statistically significant pre-/post-antiandrogen treatment increase in the PSAIDM in the subject indicates therapeutic effectiveness of the antiandrogen treatment. Often, the sequence of the extension product of the primer that is complementary to the AMDM is determined, and determining a sequence of the extension product of the primer that is complementary to the PSAIDM is determined.

In a frequent embodiment, when detecting a statistically significant pre-/post-treatment decrease in the one or more androgen modulated diagnostic marker(s), and a statistically significant pre-/post-treatment increase in the one or more prostate-specific, androgen independent, diagnostic marker(s) in the patient/human indicates that the antiandrogen therapy resulted in prostate cell death.

In another embodiment the one or more androgen modulated diagnostic marker(s) and the one or more prostate-specific, androgen independent, diagnostic marker(s) are measured in a blood or a urine sample.

In one embodiment the one or more androgen modulated diagnostic marker(s) comprise prostate specific antigen (PSA) mRNA, prostate cancer antigen 3 (PCA3), one or more fusions of an androgen regulated gene with an ETS family member gene, or a combination of two or more of these diagnostic markers.

In another embodiment the one or more androgen modulated diagnostic marker(s) comprises PCA3 and one or more fusions of an androgen regulated gene with an ETS family member gene. In a further embodiment the prostate-specific, androgen independent, diagnostic marker comprises PSMA.

In another embodiment one or more measurements of the one or more androgen modulated diagnostic marker(s) and one or more prostate-specific, androgen independent, diagnostic marker(s) are taken before antiandrogen treatment.

In another embodiment more than one measurement of the one or more androgen modulated diagnostic marker(s) and one or more prostate-specific, androgen independent, diagnostic marker(s) are taken after antiandrogen treatment. In a further embodiment the measurements taken after antiandrogen treatment are periodic measurements over time.

In another embodiment a kinetic profile of the one or more androgen modulated diagnostic marker(s) and the one or more prostate-specific, androgen independent, diagnostic marker(s) is generated based on the periodic measurements over time.

In one embodiment the one or more androgen modulated diagnostic marker(s) comprises a fusion of an androgen regulated gene with an ETS family member gene, wherein the androgen regulated gene is selected from the group consisting of TMPRSS2, PSA, SLC45A3, HERV-K_{—}22q11.23, KLK2, SNRK, Seladin-1, FKBP51, FLI35294, CANT1, DDX5, USP10, EIF4E2, RC3H2, LMAN2, MIPOL1, HERPUD1, TIA1, NUP214, DLEU2, PIK3C2A, SPOCK1, RERE, AHCYL1, ARHGAP19, BC017255, FCHO1, PAPOLA, CARM1, MGC11102, SLC4A1AP, ERCC2, PMF1, THOC6, NDUFB8, ANKRD39, C14orf124, C14orf21, and ZNF511.

In another embodiment the one or more androgen modulated diagnostic marker(s) comprises a fusion of an androgen regulated gene with an ETS family member gene, wherein the ETS family gene is selected from the group consisting of ERG, ETV1, ETV4, FLI1, ETS1, ETS2, ELK1, ETV6 (TEL1), ETV7 (TEL2), GABPα, ELF1, ETV5 (ERM), ERF, PEA3/E1AF, PU.1, ESE1/ESX, SAP1 (ELK4), ETV3 (METS), EWS/FLI1, ESE1, ESE2 (ELF5), ESE3, PDEF, NET (ELK3; SAP2), NERF (ELF2), FEV, ZDHHC7, HJURP, INPP4ASTRN4, RGS3, AP3S1, DGKB, DIRC2, XKR3, PSPC1, TEAD1, TBC1D9B, PIK3CD, RAD51C, DRG1, TMEM49, MY09B, AK7, YIPF2, BANF1, SUPT7L, KLC3, BGLAP, HCFC1R1, SEC31L2, ANKRD23, KIAA0323, CIDEB, and TUBGCP2.

In another embodiment the one or more androgen modulated diagnostic marker(s) comprise(s) a fusion of an androgen regulated gene with an ETS family member gene, wherein the androgen regulated gene comprises TMPRSS2, SLC45A3, HERV-K, C15orf21, DDX5, CANT1, FLI35294, NDRG1, HERPUD1, HNRPA2B1, ACSL3, EST14, or KLK2 and the ETS family member gene comprises ERG, ELK4, ETV1, ETV4, or ETV5.

In another embodiment the statistically significant pre-/post-treatment decrease in the one or more androgen modulated markers, and the statistically significant pre-/post-treatment increase in the one or more prostate-specific, androgen independent, diagnostic markers is determined relative to the typical variability range of these markers in cancer patients in the absence of antiandrogen treatment.

In a frequent embodiment the cancer is prostate cancer. In a related embodiment the cancer is metastatic prostate cancer.

In one embodiment the antiandrogen therapeutic or compound is selected from the group consisting of spironolactone, cyproterone, cyproterone acetate, flutamide, nilutamide, triptorelin, bicalutamide, ketoconazole, finasteride, dutasteride, DDE, bexlosteride, izonsteride, epristeride, turosteride, abiraterone, abiraterone acetate, MDV3100, BMS-641988, RD162, 3beta-hydroxy-17-(1H-benzimidazole-1-yl)androsta-5,16-diene (VN/124-1), steroidal C-17 benzoazoles and pyrazines, leuprolide, goserelin, abarelix, medroxyprogesterone, megestrol, and a combination of two or more of the preceding therapeutics.

In a frequent embodiment kits are provided for use in any of the described methods. Often these kits include one or more reagents necessary to carry out the methods, frequently also including a package insert or instructions.

### DETAILED DESCRIPTION OF THE INVENTION

### I. Definitions

Unless defined otherwise, all terms of art, notations and other scientific terms or terminology used herein have the same meaning as is commonly understood by one of ordinary skill in the art to which this disclosure belongs. Many of the techniques and procedures described or referenced herein are well understood and commonly employed using conventional methodology by those skilled in the art. As appropriate, procedures involving the use of commercially available kits and reagents are generally carried out in accordance with manufacturer defined protocols and/or parameters unless otherwise noted. All patents, applications, published applications and other publications referred to herein are incorporated by reference with regard to each issue for which they are cited, and related issues. If a definition set forth in this section is contrary to or otherwise inconsistent with a definition set forth in the patents, applications, published applications, and other publications that are herein incorporated by reference, the definition set forth in this section prevails over the definition that is incorporated herein by reference.

As used herein, “a” or “an” means “at least one” or “one or more.”

As used herein, the term “gene fusion” refers to a chimeric genomic DNA, a chimeric messenger RNA, a truncated protein or a chimeric protein resulting from the fusion of at least a portion of a first gene to at least a portion of a second gene. The gene fusion need not include entire genes or exons of genes. Exemplary gene fusions of the present invention are described, for example, in U.S. Pat. No. 7,718,369; and U.S. Patent Application Publication Nos. 2009-0208937, 2009-0239221, 2011-0028336, 2011-0065113, 2012-0015839, and 2012-039887. Gene fusions are often referred to herein through a nomenclature featuring the 5′ or N-terminal fusion or rearrangement partner listed first, separated by a colon “:” or dash “-” from the 3′ or C-terminal fusion or rearrangement partner.

As used herein, the term “transcriptional regulatory region” refers to the region of a gene comprising sequences that modulate (e.g., upregulate or down-regulate) expression of the gene. In some embodiments, the transcriptional regulatory region of a gene comprises non-coding upstream sequence of a gene, also called the 5′ untranslated region (5′UTR). In other embodiments, the transcriptional regulatory region contains sequences located within the coding region of a gene or within an intron (e.g., enhancers).

As used herein, the term “androgen regulated gene” refers to a gene or portion of a gene whose expression is induced or repressed by an androgen (e.g., testosterone). The promoter region of an androgen regulated gene may contain an “androgen response element” that interacts with androgens or androgen signaling molecules (e.g., downstream signaling molecules).

As used herein, the terms “detect,” “detecting,” or “detection” may describe either the general act of discovering or discerning or the specific observation of a molecule or composition, whether directly or indirectly labeled with a detectable label.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethy 1-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (nRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer.” Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the term “significantly” or “statistically significant” refers to a p-value of less than 0.05, e.g., a p-value of less than 0.025 or a p-value of less than 0.01, using an appropriate measure of statistical significance, e.g., a one-tailed two sample t-test. In certain embodiments a statistically significant change in the measured pre-/post-therapy levels of the androgen regulated marker(s) or the prostate-specific, androgen independent, marker(s) represents a change of at least one standard deviation from the level of the same markers in an untreated control. In a frequent embodiment a statistically significant change in the measured pre-/post-therapy levels of the androgen regulated marker(s) or the prostate-specific, androgen independent, marker(s) represents a change of about 2, about 3, or about 4 or more standard deviations from the level of the same markers in an untreated control. Often a statistically significant change in the measured pre-/post-therapy levels of the androgen regulated marker(s) or the prostate-specific, androgen independent, marker(s) represents a several fold change in the level of the markers compared with the level of the same markers in an untreated control. In any event, the determination of whether a statistically significant change has occurred in the measured pre-/post-therapy levels of one or more androgen regulated marker or one or more prostate-specific, androgen independent, marker is most often determined with reference to the level of the same marker in an untreated control sample, taking into account the typical assay variability (i.e., standard deviation) of that marker over multiple samplings of an untreated control sample or a normal sample.

Any patient sample suspected of containing the markers of the present invention may be tested according to the methods of the present disclosure. By way of non-limiting example, the sample may be tissue (e.g., a prostate biopsy sample or a tissue sample obtained by prostatectomy), blood, urine, semen, cells, cell secretions or a fraction thereof (e.g., plasma, serum, exosomes, urine supernatant, or urine cell pellet). A urine sample is often collected immediately following an attentive digital rectal examination (DRE), which causes prostate cells from the prostate gland to shed into the urinary tract.

The patient sample may require preliminary processing designed to isolate or enrich the sample for the markers or cells that contain the markers. A variety of techniques known to those of ordinary skill in the art may be used for this purpose, including but not limited to: centrifugation; immunocapture; cell lysis; and, nucleic acid target capture (See, e.g., EP Pat. No. 1409727).

### II. Androgen Modulated Diagnostic Markers

A. Prostate Surface Antigen

Despite the fact that varying blood or urine PSA levels can be the result from a variety of different causes, it is nonetheless the basis for primary screening for prostate cancer. Measurement of total PSA (tPSA) as a diagnostic assay to predict prostate cancer has been in use since the early 1990s. Levels of 4 ng/ml or greater in blood serum are considered abnormal and predictive of prostate cancer. However, the sensitivity of such elevated tPSA levels is only 79%; thus leaving 21% of patients with prostate cancer undetected. The specificity for all tPSA values of 4 ng/ml or greater is very poor. In addition, estimates of specificity for tPSA levels >4.0 ng/ml are reported to be in the range of 20% to 59%, averaging around 33%. The vast majority of false positives are ultimately shown to be benign prostatic hyperplasia (BPH). The specificity is lowest for modestly elevated tPSA, in the low “gray zone” of 4 to 10 ng/ml. This low level of specificity often leads to additional, more invasive and costly, diagnostic procedures such as transrectal ultrasounds and prostate biopsies. Such tests when unnecessary can also be stressful for the patient.

Because of the shortcomings of tPSA, research has been focused on attempting to develop PSA derivatives to increase the sensitivity and specificity of this general diagnostic approach. One modification is free PSA (fPSA), which was FDA approved in 1998. PSA in serum can be found either in an unbound form or complexed with circulating protease inhibitors, most commonly with alpha-1-antitrypsin (ACT). Clinicians have shown that the proportion of PSA bound to ACT was significantly higher in men with prostate cancer than in unaffected men or those with BPH. As a guideline, if 25% or less of total PSA is free, this is an indicator of possible prostate cancer. The fPSA assay was approved for use in men with tPSA's for 4 to 10 ng/ml. Thus, the fPSA assay was positioned to improve the specificity over that of tPSA alone. However, the predictive power of the fPSA test is not as good in people with really low or really high tPSA levels. Very low tPSA, regardless of measured fPSA, is predictive of not having cancer, while the converse may be true with very high tPSA levels. The diagnostic usefulness of fPSA is relatively limited as it can be associated with either BPH or prostate cancer. The use of fPSA in combination with tPSA has been shown to reduce the number of unnecessary biopsies by about 20%.

PSA concentrations decrease as a result of androgen deprivation therapy. See, e.g., de Bono et al., New. Engl. J. Med., 2011, 364(21):1995-2005; Mohler et al., Clin. Cancer. Res., 2004, 10:440-448. Similarly, inhibition of androgen biosynthesis results in a decrease of PSA concentrations, for example by way of administration of abiraterone acetate. See id.

B. Prostate Cancer Antigen 3 (PCA3)

Another prostate cancer marker, PCA3, was discovered several years ago by differential display analysis intended to highlight genes associated with prostate cancer development. PCA3 is located on chromosome 9 and composed of four exons. It encodes at least four different transcripts which are generated by alternative splicing and polyadenylation. However, PCA3 likely functions as a noncoding RNA. See, e.g., Bussemakers et al., Cancer Res., 1999, 59(23):5975-9; Schalken et al., Urology, 2003, 62(Suppl 5A):34-43. By RT-PCR analysis, PCA3 expression was found to be limited to the prostate and absent in all other tissues tested, including testis, ovary, breast and bladder. Northern blot analysis showed that PCA3 is highly expressed in the vast majority of prostate cancers examined (47 out of 50) whereas no or very low expression is detected in BPH or normal prostate cells from the same patients (Bussemakers et al., Cancer Res., 1999, 59(23):5975-9, incorporated herein by reference). As such, PCA3 has been demonstrated to be prostate cancer specific, and highly overexpressed in prostate tumors. See, e.g., Schalken et al., Urology, 2003, 62(Suppl 5A):34-43.

PCA3 has been shown to be androgen-dependent in vitro in that levels of PCA3 decrease in the absence of androgen, and vice-versa. See, e.g., Shaw & Prowse, Eur. Urol., 2007, 51:856-864.

C. Androgen Regulated Gene (ARG):E-Twenty Six (ETS) Gene Fusions

Recently fusions between the androgen-regulated transmembrane protease serine 2 gene, TMPRSS2, and E twenty-six (ETS) transcription factors were discovered in prostate cancer (Tomlins et al., Science, 2005, 310:644-8). It was discovered that, in fact, a whole class of novel 5′ and 3′ fusion partners between an androgen regulated gene (ARG) and an ETS family member were expressed in prostate cancer (see, e.g., Tomlins et al., Cancer Res., 2006, 66:3396-400; Helgeson et al., Cancer Res., 2008, 68:73-80; Rickman et al., Cancer Res., 2009, 69:2734-8; Tomlins et al., Nature, 2007, 448:595-9; Tomlins et al., Eur. Urol., 2009, 56(2):275-86; U.S. Pat. No. 7,718,369; U.S. Patent Application Publication No. 2009-0208937; U.S. Patent Application Publication No. 2009-0239221; PCT Publication No. WO2010-081001; and PCT Publication No. WO2010-096660). TMPRSS2:ERG is the most common fusion, which is present in approximately 50% of prostate-specific antigen (PSA)-screened localized prostate cancers and in 15-35% of population-based cohorts. These fusions can be detected noninvasively in the urine of men with prostate cancer, with a >90% specificity rate in PSA-screened patient populations. Reports from untreated population-based patient groups suggest an association between ETS fusions and cancer-specific death and metastatic spread.

Methods of detecting presence of a TMPRSS2-ERG gene fusion are known to one of skill in the art. In some examples, the TMPRSS2-ERG gene fusion is detectable as DNA, RNA or protein. In one example, the presence of TMPRSS2-ERG gene fusion is determined by detecting a genomic DNA having a 5′ portion from a TMPRSS2 gene (such as a transcriptional regulatory portion of a TMPRSS2 gene) and a 3′ portion from an ERG gene, for example by in situ hybridization (for example, fluorescent in situ hybridization or colorimetric in situ hybridization), Southern blot, or DNA sequencing. In other examples, the presence of a TMPRSS2-ERG gene fusion is determined by detecting the presence of a chimeric mRNA having a 5′ portion from a TMPRSS2 gene (such as a transcriptional regulatory region of the TMPRSS2 gene) and a 3′ portion from the ERG gene, for example by Northern blot, polymerase chain reaction (such as reverse transcription PCR), transcription mediated amplification, or nucleic acid sequencing. In still further examples the presence of a TMRPSS2-ERG gene fusion is determined by detecting the presence of an amino-terminally truncated ERG protein resulting from the fusion of the TMPRSS2 gene to ERG gene or detecting a chimeric protein having an amino-terminal portion from the TMPRSS2 gene and a carboxyl-terminal portion from the ERG gene, for example by immunoassay (such as Western blot or immunohistochemistry).

Similar methods are utilized to identify the whole class of ARG:ETS fusions, for example, as described in U.S. Pat. No. 7,718,369; and U.S. Patent Application Publication Nos. 2009-0208937, 2009-0239221, 2011-0028336, 2011-0065113, 2012-0015839, and 2012-039887. Though not intended to be a comprehensive list of prostate cancer specific gene fusions known in the art, these patents and applications describe additional fusions including, for example, TMPRSS2:ERG, TMPRSS2:ETV1, TMPRSS2:ETV4, TMPRSS2:FLI1, TMPRSS2:ETV5, HERV-K_{—}22q11.23:ETV1, SLC45A3:ETV1, SLC45A3:ELK4, SLC45A3:ETV5, HERPUD1:ERG, SLC45A3:ERG, FLI35294:ETV1, CANT1:ETV4, NDRG1:ERG, C15ORF21:ETV1, HNRPA2B1:ETV1, ACSL3:ETV1, ETS14:ETV1, HERVK17:ETV1, DDX5:ETV4, CANT1:ETV4, KLK2:ETV4, USP10:ZDHHC7, EIF4E2:HJURP, HJURP:INPP4, ASTRN4:GPSN2, RC3H2:RGS3, LMAN2:AP3S1, ZNF649-ZNF577, MIPOL1:DGKB, TIA1:DIRC2, NUP214:XKR3, DLEU2:PSPC1, PIK3C2A:TEAD1, SPOCK1:TBC1D9B, RERE:PIK3CD, AHCYL1:RAD51C, ARHGAP19:DRG1, BC017255:TMEM49, FCHO1:MY09B, PAPOLA:AK7, CARM1:YIPF2, MGC11102:BANF1, SLC4A1AP:SUPT7L, ERCC2:KLC3, PMF1:BGLAP, THOC6:HCFC1R1, NDUFB8:SEC31L2, ANKRD39:ANKRD23, C14orf124:KIAA0323, C14orf21:CIDEB, and ZNF511:TUBGCP2.

The TMPRSS2 gene itself is strongly induced by androgen. See, e.g., Tomlins et al., Science, 2005, 310:644-648. See id. In addition, when the ERG oncogene is fused with the TMPRSS2 gene it has also been shown to be overexpressed in a significant percentage of prostate canser. See id.; Attard et al., Cancer Res., 2009, 69(7):2912-2918. Similarly, the gene fusions SLC45A3:ERG, NDRG1:ERG, TMPRSS2:ETV1, HERV-K_{—}22q11.23:ETV1, SLC45A3:ETV1, TMPRSS2:ETV4, KLK2:ETV4, CANT1:ETV4, TMPRSS2:ETV5, SLC45A3:ETV5, and FLI35294:ETV1 are androgen regulated. See, e.g., Pflueger et al., Neoplasia, 2009, 11(8):804-811; Kumar-Sinha et al., Nat. Rev. Cancer, 2008, 8(7):497-511; Tomlins et al., Cancer Res., 2006, 66:3396-3400; Helgeson et al., Cancer Res., 2008, 68(1):73-80; Han et al., Cancer Res., 2008, 68(18):7629-7637.

### III. Prostate Specific, Androgen Independent, Diagnostic Markers

A. Prostate Specific Membrane Antigen (PSMA)

PSMA is an integral membrane protein that is highly specific for the prostate. See, e.g., Murphy et al., Cancer, 1998, 83:2259-2269. PSMA is expressed in benign prostate tissue, but is upregulated and strongly expressed in primary and metastatic prostate cancer. In particular, PSMA is expressed in all primary prostate cancer and 57.7%-100% of metastatic prostatic carcinomas (Sweat et al., Urology, 198, 52:637-40). As such, PSMA is prostate tissue specific, similar to PSA, PCA3, and ARG:ETS gene fusions. However, PSMA gene expression is correlated with androgen-independent prostate cancer (see, e.g., Kim et al., Urologic Oncology, 2000, 5:97-103) and has been found to be upregulated or unaffected in patients that have been subject to medical or surgical castration or androgen-deprivation therapy (Wright et al., Urology, 1996, 48:326-334).

In an assay of 236 individual serum samples, it was determined that for normal males under 50 years of age, PSMA ranged from zero (i.e., no detectable PSMA) to 9.564, having a mean value of 1.56. In normal males over the age of 50, the PSMA range and mean serum levels were 0-16.834 and 3.48, respectively (see Beckett et al., Clin. Cancer Res., 1999, 5:4034-40, incorporated herein by reference). In the same study, men with benign prostatic hyperplasia, prostatitis, and stage T1/T2 prostate cancer showed PSMA levels varying from 0-17.266, with a mean value of 1.75, 2.64, and 1.7, respectively. In stage T3 prostate cancer this study found serum PSMA levels to vary from 0.25-31.5, with a mean of 6.82.

RNA transcripts in urine have also been evaluated for PSMA presence in prostate cancer patients. See, e.g., Rigau et al., Prostate, December 2011, 71(16):1736-45. Certain embodiments of the present invention rely on evaluation of PSMA polynucleotides such as RNA transcripts in urine.

### IV. Antiandrogen therapy

Antiandrogens include any group of hormone receptor antagonist compounds that are capable of preventing or inhibiting the biological effects of androgens. Typically, antiandrogens work by obstructing the biological pathway induced by androgens such as by blocking, or competing for, the appropriate androgen receptors, by modulating androgen levels or by inhibiting steroid hormone biosynthesis. Since androgens, such as testosterone, bind prostate cell receptors and fuel prostate cancer cell growth, reducing or eliminating their ability to perform this task has, as discussed above, been utilized in prostate cancer treatment.

Suitable antiandrogen compounds include any antiandrogen or androgen antagonist therapeutic including spironolactone, cyproterone, cyproterone acetate, flutamide, nilutamide, triptorelin, bicalutamide, ketoconazole, finasteride, dutasteride, DDE, bexlosteride, izonsteride, epristeride, turosteride, abiraterone, abiraterone acetate, leuprolide, goserelin, abarelix, medroxyprogesterone, megestrol, MDV3100, BMS-641988, RD162, and CYP17 inhibitors/antiandrogens such as 3beta-hydroxy-17-(1H-benzimidazole-1-yl)androsta-5,16-diene (VN/124-1), and other steroidal C-17 benzoazoles and pyrazines including those described, for example, in U.S. Patent Application Publication No. 20100137269. Suitable antiandrogen compounds and androgen antagonists include compounds that target androgen production and androgen receptor mediated signaling, such as those known in the art and those noted in, for example, Molina & Belldegrun, J. Urology, 2011, 185:787-794. This list of the types of antiandrogens and androgen antagonists is not intended to be limiting and is provided by way of example only. Any compounds or therapies exhibiting an antiandrogen action, androgen antagonistic activity, androgen production suppressive activity, or adversely affecting or blocking androgen receptor mediated signaling are intended to be encompassed by the phrase “antiandrogenantiandrogen therapy” or “antiandrogen compound.”

### V. Antiandrogen Effect on Androgen Regulated, and Androgen Independent, Genes

The inventors have determined that conventional biological markers useful to detect prostate cancer may not provide the most reliable indicators of the therapeutic efficacy of an antiandrogen therapeutic. For example, as discussed above, androgen regulated diagnostic markers such as PSA mRNA, PCA3, and ARG:ETS fusions are valuable to assess the effect of antiandrogens on the inhibition of gene expression. This is the case since mRNA can be monitored for each of these genes after antiandrogen administration. However, the effect of antiandrogens on actual tumor cell death is generally not evaluated by assessing these genes because the expression of each of these genes is androgen dependent. In other words, decreased expression of these genes will generally occur when an antiandrogenantiandrogen therapy is administered, regardless of tumor cell kill. A decrease in mRNA levels of these types of androgen-dependent genes shows only that the gene or gene fusion expression has decreased or non-detectable expression of the particular gene or gene-fusion. In such cases prostate cancer cells expressing these genes may be alive, but expression of these androgen-dependent genes has decreased.

In contrast, PSMA expression, which is highly prostate-specific, but androgen independent, is not inhibited by antiandrogenantiandrogen therapeutics. Thus, when an antiandrogen therapeutic is administered to a subject, expression levels of PSMA (among other prostate specific, androgen independent markers) should not be affected. However, the present inventors have realized that increased levels of PSMA (among other prostate specific, androgen independent markers) can be detected in urine or blood in a subject following antiandrogen therapy, when that therapy results in prostate cell death. Prostate cell death results in the release of PSMA, for example, into fluids and tissues surrounding the dead cells. Though not intending to be bound by any particular theory related to the transport of PSMA in bodily fluids, nucleic acids derived from dead prostate cancer cells or cell fragments generally will make their way into urine or blood where they can be assayed in samples of these fluids. Frequently, in localized prostate cancer confined to prostate tissues, PSMA nucleic acids will be transported via the prostatatic ductal system in the prostate to the urethra. A DRE exam will frequently aid the process of biomarker (including, for example, PSA, PCA3, ARG:ETS fusions, and PSMA) transport to the urine. If prostate cancer has metastasized, or if the patent has been subject to prostatectomy, blood may provide a preferable, alternate, or secondary sample source since tumor cells may not have direct access to the urinary tract for assay in urine samples.

### VII. Marker Panels

Though provided only by way of example, and not intending to be limiting, the following Table 1 outlines certain contemplated diagnostic marker combinations, according the present invention, that one can utilize in accordance with the presently described methods. The left column provides the sequentially numbered indication of where each marker is positioned in the Table. The middle column indicates the identified androgen modulated diagnostic marker or marker combination to be included together with a prostate specific, androgen independent diagnostic marker in a diagnostic. The right column indicates the identified prostate specific, androgen independent diagnostic marker to be included together with an androgen modulated diagnostic marker or markers. The reference to “or other PSAI marker” in the right column refers to single PSAIDM as well as PSAIDM combinations. For purposes of brevity only, where row two of Table 1 includes only a specific combination of numerals, this refers to the androgen modulated diagnostic markers set forth in the sequentially numbered positions in the table corresponding to each number (for example, “1, 2” refers to “PSA+PCA3”).

TABLE 1

| | | Prostate Specific, |
|---|---|---|
| | Androgen | Androgen |
| | Modulated | Independent, |
| | Diagnostic | Diagnostic |
| No. | Marker | Marker (PSAIM) |
| 1 | PSA | PSMA or other |
| | | PSAI marker |
| 2 | PCA3 | PSMA or other |
| | | PSAI marker |
| 3 | TMPRSS2:ERG | PSMA or other |
| | | PSAI marker |
| 4 | SLC45A3:ERG | PSMA or other |
| | | PSAI marker |
| 5 | SLC45A3:ELK4 | PSMA or other |
| | | PSAI marker |
| 6 | HERPUD1:ERG | PSMA or other |
| | | PSAI marker |
| 7 | NDRG1:ERG | PSMA or other |
| | | PSAI marker |
| 8 | TMPRSS:ETV1 | PSMA or other |
| | | PSAI marker |
| 9 | SLC45A3:ETV1 | PSMA or other |
| | | PSAI marker |
| 10 | HERV-K:ETV1 | PSMA or other |
| | | PSAI marker |
| 11 | C15ORF21:ETV1 | PSMA or other |
| | | PSAI marker |
| 12 | HNRPA2B1:ETV1 | PSMA or other |
| | | PSAI marker |
| 13 | FLJ35294:ETV1 | PSMA or other |
| | | PSAI marker |
| 14 | ACSL3:ETV1 | PSMA or other |
| | | PSAI marker |
| 15 | EST14:ETV1 | PSMA or other |
| | | PSAI marker |
| 16 | HERVk17:ETV1 | PSMA or other |
| | | PSAI marker |
| 17 | TMPRSS2:ETV4 | PSMA or other |
| | | PSAI marker |
| 18 | DDX5:ETV4 | PSMA or other |
| | | PSAI marker |
| 19 | CANT1:ETV4 | PSMA or other |
| | | PSAI marker |
| 20 | KLK2:ETV4 | PSMA or other |
| | | PSAI marker |
| 21 | TMPRSS2:ETV5 | PSMA or other |
| | | PSAI marker |
| 22 | SLC45A3:ETV5 | PSMA or other |
| | | PSAI marker |
| 23 | ESRP1:CRAF | PSMA or other |
| | | PSAI marker |
| 24 | SLC45A3:BRAF | PSMA or other |
| | | PSAI marker |
| 25 | CRAF:ESRP1 | PSMA or other |
| | | PSAI marker |
| 26 | 1, 2 | PSMA or other |
| | | PSAI marker |
| 27 | 1, 3 | PSMA or other |
| | | PSAI marker |
| 28 | 2, 3 | PSMA or other |
| | | PSAI marker |
| 29 | 1, 2, 3 | PSMA or other |
| | | PSAI marker |
| 30 | 1, 4 | PSMA or other |
| | | PSAI marker |
| 31 | 2, 4 | PSMA or other |
| | | PSAI marker |
| 32 | 3, 4 | PSMA or other |
| | | PSAI marker |
| 33 | 1, 2, 4 | PSMA or other |
| | | PSAI marker |
| 34 | 1, 2, 3, 4 | PSMA or other |
| | | PSAI marker |
| 35 | 1, 5 | PSMA or other |
| | | PSAI marker |
| 36 | 2, 5 | PSMA or other |
| | | PSAI marker |
| 37 | 3, 5 | PSMA or other |
| | | PSAI marker |
| 38 | 4, 5 | PSMA or other |
| | | PSAI marker |
| 39 | 1, 2, 5 | PSMA or other |
| | | PSAI marker |
| 40 | 1, 2, 3, 5 | PSMA or other |
| | | PSAI marker |
| 41 | 1, 2, 3, 4, 5 | PSMA or other |
| | | PSAI marker |
| 42 | 1, 8 | PSMA or other |
| | | PSAI marker |
| 43 | 2, 8 | PSMA or other |
| | | PSAI marker |
| 44 | 3, 8 | PSMA or other |
| | | PSAI marker |
| 45 | 4, 8 | PSMA or other |
| | | PSAI marker |
| 46 | 5, 8 | PSMA or other |
| | | PSAI marker |
| 47 | 1, 2, 8 | PSMA or other |
| | | PSAI marker |
| 48 | 1, 2, 3, 8 | PSMA or other |
| | | PSAI marker |
| 49 | 1, 2, 3, 4, 8 | PSMA or other |
| | | PSAI marker |
| 50 | 1, 2, 3, 4, 5, 8 | PSMA or other |
| | | PSAI marker |
| 51 | 1, 9 | PSMA or other |
| | | PSAI marker |
| 52 | 2, 9 | PSMA or other |
| | | PSAI marker |
| 53 | 3, 9 | PSMA or other |
| | | PSAI marker |
| 54 | 4, 9 | PSMA or other |
| | | PSAI marker |
| 55 | 5, 9 | PSMA or other |
| | | PSAI marker |
| 56 | 8, 9 | PSMA or other |
| | | PSAI marker |
| 57 | 1, 2, 9 | PSMA or other |
| | | PSAI marker |
| 58 | 1, 2, 3, 9 | PSMA or other |
| | | PSAI marker |
| 59 | 1, 2, 3, 4, 9 | PSMA or other |
| | | PSAI marker |
| 60 | 1, 2, 3, 4, 5, 9 | PSMA or other |
| | | PSAI marker |
| 61 | 1, 2, 3, 4, 5, 8, 9 | PSMA or other |
| | | PSAI marker |
| 62 | 1, 17 | PSMA or other |
| | | PSAI marker |
| 63 | 2, 17 | PSMA or other |
| | | PSAI marker |
| 64 | 3, 17 | PSMA or other |
| | | PSAI marker |
| 65 | 4, 17 | PSMA or other |
| | | PSAI marker |
| 66 | 5, 17 | PSMA or other |
| | | PSAI marker |
| 67 | 8, 17 | PSMA or other |
| | | PSAI marker |
| 68 | 9, 17 | PSMA or other |
| | | PSAI marker |
| 69 | 1, 2, 17 | PSMA or other |
| | | PSAI marker |
| 70 | 1, 2, 3, 17 | PSMA or other |
| | | PSAI marker |
| 71 | 1, 2, 3, 4, 17 | PSMA or other |
| | | PSAI marker |
| 72 | 1, 2, 3, 4, 5, 17 | PSMA or other |
| | | PSAI marker |
| 73 | 1, 2, 3, 4, 5, 8, 17 | PSMA or other |
| | | PSAI marker |
| 74 | 1, 2, 3, 4, 5, 8, 9, | PSMA or other |
| | 17 | PSAI marker |
| 75 | 1, 21 | PSMA or other |
| | | PSAI marker |
| 76 | 2, 21 | PSMA or other |
| | | PSAI marker |
| 77 | 3, 21 | PSMA or other |
| | | PSAI marker |
| 78 | 4, 21 | PSMA or other |
| | | PSAI marker |
| 79 | 5, 21 | PSMA or other |
| | | PSAI marker |
| 80 | 8, 21 | PSMA or other |
| | | PSAI marker |
| 81 | 9, 21 | PSMA or other |
| | | PSAI marker |
| 82 | 17, 21 | PSMA or other |
| | | PSAI marker |
| 83 | 1, 2, 21 | PSMA or other |
| | | PSAI marker |
| 84 | 1, 2, 3, 21 | PSMA or other |
| | | PSAI marker |
| 85 | 1, 2, 3, 4, 21 | PSMA or other |
| | | PSAI marker |
| 86 | 1, 2, 3, 4, 5, 21 | PSMA or other |
| | | PSAI marker |
| 87 | 1, 2, 3, 4, 5, 8, 21 | PSMA or other |
| | | PSAI marker |
| 88 | 1, 2, 3, 4, 5, 8, 9, | PSMA or other |
| | 21 | PSAI marker |
| 89 | 1, 2, 3, 4, 5, 8, 9, | PSMA or other |
| | 17, 21 | PSAI marker |
| 90 | 1, 22 | PSMA or other |
| | | PSAI marker |
| 91 | 2, 22 | PSMA or other |
| | | PSAI marker |
| 92 | 3, 22 | PSMA or other |
| | | PSAI marker |
| 93 | 4, 22 | PSMA or other |
| | | PSAI marker |
| 94 | 5, 22 | PSMA or other |
| | | PSAI marker |
| 95 | 8, 22 | PSMA or other |
| | | PSAI marker |
| 96 | 9, 22 | PSMA or other |
| | | PSAI marker |
| 97 | 17, 22 | PSMA or other |
| | | PSAI marker |
| 98 | 21, 22 | PSMA or other |
| | | PSAI marker |
| 99 | 1, 2, 22 | PSMA or other |
| | | PSAI marker |
| 100 | 1, 2, 3, 22 | PSMA or other |
| | | PSAI marker |
| 101 | 1, 2, 3, 4, 22 | PSMA or other |
| | | PSAI marker |
| 102 | 1, 2, 3, 4, 5, 22 | PSMA or other |
| | | PSAI marker |
| 103 | 1, 2, 3, 4, 5, 8, 22 | PSMA or other |
| | | PSAI marker |
| 104 | 1, 2, 3, 4, 5, 8, 9, | PSMA or other |
| | 22 | PSAI marker |
| 105 | 1, 2, 3, 4, 5, 8, 9, | PSMA or other |
| | 17, 22 | PSAI marker |
| 106 | 1, 2, 3, 4, 5, 8, 9, | PSMA or other |
| | 17, 21, 22 | PSAI marker |
| 107 | | PSMA or other |
| | | PSAI marker |
| 108 | 1, 23 | PSMA or other |
| | | PSAI marker |
| 109 | 2, 23 | PSMA or other |
| | | PSAI marker |
| 110 | 3, 23 | PSMA or other |
| | | PSAI marker |
| 111 | 4, 23 | PSMA or other |
| | | PSAI marker |
| 112 | 5, 23 | PSMA or other |
| | | PSAI marker |
| 113 | 8, 23 | PSMA or other |
| | | PSAI marker |
| 114 | 9, 23 | PSMA or other |
| | | PSAI marker |
| 115 | 17, 23 | PSMA or other |
| | | PSAI marker |
| 116 | 21, 23 | PSMA or other |
| | | PSAI marker |
| 117 | 1, 2, 23 | PSMA or other |
| | | PSAI marker |
| 118 | 1, 2, 3, 4, 5, 8, 9, | PSMA or other |
| | 17, 21, 23 | PSAI marker |
| 119 | 1, 2, 3, 23 | PSMA or other |
| | | PSAI marker |
| 120 | 1, 2, 3, 4, 23 | PSMA or other |
| | | PSAI marker |
| 121 | 1, 2, 3, 4, 5, 23 | PSMA or other |
| | | PSAI marker |
| 122 | 1, 2, 3, 4, 5, 8, 23 | PSMA or other |
| | | PSAI marker |
| 123 | 1, 2, 3, 4, 5, 8, 9, | PSMA or other |
| | 23 | PSAI marker |
| 124 | 1, 2, 3, 4, 5, 8, 9, | PSMA or other |
| | 17, 23 | PSAI marker |
| 125 | 1, 2, 3, 4, 5, 8, 9, | PSMA or other |
| | 17, 21, 23 | PSAI marker |
| 126 | 1, 2, 3, 4, 5, 8, 9, | PSMA or other |
| | 17, 21, 22, 23 | PSAI marker |
| 127 | 1, 24 | PSMA or other |
| | | PSAI marker |
| 128 | 2, 24 | PSMA or other |
| | | PSAI marker |
| 129 | 3, 24 | PSMA or other |
| | | PSAI marker |
| 130 | 4, 24 | PSMA or other |
| | | PSAI marker |
| 131 | 5, 24 | PSMA or other |
| | | PSAI marker |
| 132 | 8, 24 | PSMA or other |
| | | PSAI marker |
| 133 | 9, 24 | PSMA or other |
| | | PSAI marker |
| 134 | 17, 24 | PSMA or other |
| | | PSAI marker |
| 135 | 21, 24 | PSMA or other |
| | | PSAI marker |
| 136 | 1, 2, 24 | PSMA or other |
| | | PSAI marker |
| 137 | 1, 2, 3, 24 | PSMA or other |
| | | PSAI marker |
| 138 | 1, 2, 3, 4, 24 | PSMA or other |
| | | PSAI marker |
| 139 | 1, 2, 3, 4, 5, 24 | PSMA or other |
| | | PSAI marker |
| 140 | 1, 2, 3, 4, 5, 8, 24 | PSMA or other |
| | | PSAI marker |
| 141 | 1, 2, 3, 4, 5, 8, 9, | PSMA or other |
| | 24 | PSAI marker |
| 142 | 1, 2, 3, 4, 5, 8, 9, | PSMA or other |
| | 17, 24 | PSAI marker |
| 143 | 1, 2, 3, 4, 5, 8, 9, | PSMA or other |
| | 17, 21, 24 | PSAI marker |
| 144 | 1, 2, 3, 4, 5, 8, 9, | PSMA or other |
| | 17, 21, 22, 24 | PSAI marker |
| 145 | 1, 2, 3, 4, 5, 8, 9, | PSMA or other |
| | 17, 21, 22, 23, 24 | PSAI marker |
| 146 | 1, 25 | PSMA or other |
| | | PSAI marker |
| 147 | 2, 25 | PSMA or other |
| | | PSAI marker |
| 148 | 3, 25 | PSMA or other |
| | | PSAI marker |
| 149 | 4, 25 | PSMA or other |
| | | PSAI marker |
| 150 | 5, 25 | PSMA or other |
| | | PSAI marker |
| 151 | 8, 25 | PSMA or other |
| | | PSAI marker |
| 152 | 9, 25 | PSMA or other |
| | | PSAI marker |
| 153 | 17, 25 | PSMA or other |
| | | PSAI marker |
| 154 | 21, 25 | PSMA or other |
| | | PSAI marker |
| 155 | 1, 2, 25 | PSMA or other |
| | | PSAI marker |
| 156 | 1, 2, 3, 4, 5, 8, 9, | PSMA or other |
| | 17, 21, 25 | PSAI marker |
| 157 | 1, 2, 3, 25 | PSMA or other |
| | | PSAI marker |
| 158 | 1, 2, 3, 4, 25 | PSMA or other |
| | | PSAI marker |
| 159 | 1, 2, 3, 4, 5, 25 | PSMA or other |
| | | PSAI marker |
| 160 | 1, 2, 3, 4, 5, 8, 25 | PSMA or other |
| | | PSAI marker |
| 161 | 1, 2, 3, 4, 5, 8, 9, | PSMA or other |
| | 25 | PSAI marker |
| 162 | 1, 2, 3, 4, 5, 8, 9, | PSMA or other |
| | 17, 25 | PSAI marker |
| 163 | 1, 2, 3, 4, 5, 8, 9, | PSMA or other |
| | 17, 21, 25 | PSAI marker |
| 164 | 1, 2, 3, 4, 5, 8, 9, | PSMA or other |
| | 17, 21, 22, 25 | PSAI marker |
| 165 | 1, 2, 3, 4, 5, 8, 9, | PSMA or other |
| | 17, 21, 22, 23, 25 | PSAI marker |
| 166 | 1, 2, 3, 4, 5, 8, 9, | PSMA or other |
| | 17, 21, 22, 23, 24, | PSAI marker |
| | 25 | |
| 167 | Any one or more | PSMA or other |
| | of 1-22 with any | PSAI marker |
| | one or more of | |
| | 23-25 | |
| 168 | Any one or more | PSMA or other |
| | of 23-25 | PSAI marker |
| 169 | Any one or more | PSMA or other |
| | of 1-5 with any | PSAI marker |
| | one or more of | |
| | 23-25 | |
| 170 | Any one or more | PSMA or other |
| | of 1-25 | PSAI marker |

### VII. DNA and RNA Detection

The markers of the present disclosure may be detected using a variety of nucleic acid techniques known to those of ordinary skill in the art, including but not limited to: nucleic acid sequencing; nucleic acid hybridization; and, nucleic acid amplification. A variety of these techniques are briefly outlined below.

A. Sequencing

Illustrative non-limiting examples of nucleic acid sequencing techniques include, but are not limited to, chain terminator (Sanger) sequencing and dye terminator sequencing, or high throughput sequencing methods. The present disclosure is not intended to be limited to any particular methods of sequencing.

Chain terminator sequencing uses sequence-specific termination of a DNA synthesis reaction using modified nucleotide substrates. Extension is initiated at a specific site on the template DNA by using a short radioactive, or other labeled, oligonucleotide primer complementary to the template at that region. The oligonucleotide primer is extended using a DNA polymerase, standard four deoxynucleotide bases, and a low concentration of one chain terminating nucleotide, most commonly a di-deoxynucleotide. This reaction is repeated in four separate tubes with each of the bases taking turns as the di-deoxynucleotide. Limited incorporation of the chain terminating nucleotide by the DNA polymerase results in a series of related DNA fragments that are terminated only at positions where that particular di-deoxynucleotide is used. For each reaction tube, the fragments are size-separated by electrophoresis in a slab polyacrylamide gel or a capillary tube filled with a viscous polymer. The sequence is determined by reading which lane produces a visualized mark from the labeled primer as you scan from the top of the gel to the bottom.

Dye terminator sequencing alternatively labels the terminators. Complete sequencing can be performed in a single reaction by labeling each of the di-deoxynucleotide chain-terminators with a separate fluorescent dye, which fluoresces at a different wavelength.

A variety of nucleic acid sequencing methods are contemplated for use in the methods of the present disclosure including, for example, chain terminator (Sanger) sequencing, dye terminator sequencing, high-throughput sequencing methods; and next-generation sequencing methods. Many of these sequencing methods are known in the art. See, e.g., Sanger et al., Proc. Natl. Acad. Sci. USA, 1997, 74:5463-5467; Maxam et al., Proc. Natl. Acad. Sci. USA, 1977, 74:560-564; Drmanac, et al., Nat. Biotechnol., 1988, 16:54-58; Kato, Int. J. Clin. Exp. Med., 2009, 2:193-202; Ronaghi et al., Anal. Biochem., 1996, 242:84-89; Margulies et al., Nature, 2005, 437:376-380; Ruparel et al., Proc. Natl. Acad. Sci. USA, 2005, 102:5932-5937, and Harris et al., Science, 2008, 320:106-109; Levene et al., Science, 2003, 299:682-686; Korlach et al., Proc. Natl. Acad. Sci. USA, 2008, 105:1176-1181; Branton et al., Nat. Biotechnol., 2008, 26(10):1146-53; Eid et al., Science, 2009, 323:133-138; Metzker, Nature Reviews, 2010, 11:31-46; Blow, Nature Methods, 2008, 5(3):267-72; Rothberg & Leamon, Nature Biotechnology, 2008, 26(10):1117-24; see also U.S. Pat. Nos. 7,008,766, 7,888,073, 7,939,264, and 7,608,397; and U.S. Patent Application Publication No. 2004-241678.

B. Hybridization

Illustrative non-limiting examples of nucleic acid hybridization techniques include, but are not limited to, in situ hybridization (ISH), microarray, and Southern or Northern blot.

In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH is used to measure and localize mRNAs and other transcripts within tissue sections or whole mounts. Sample cells and tissues are usually treated to fix the target transcripts in place and to increase access of the probe. The probe hybridizes to the target sequence at elevated temperature, and then the excess probe is washed away. The probe that was labeled with radio-, fluorescent- or antigen-labeled bases is localized and quantitated in the tissue using autoradiography, fluorescence microscopy or immunohistochemistry. ISH can also use two or more probes, labeled with radioactivity or the other non-radioactive labels, to simultaneously detect two or more transcripts.

1. FISH

In some embodiments, fusion sequences are detected using fluorescence in situ hybridization (FISH). The preferred FISH assays for methods of embodiments of the present disclosure utilize bacterial artificial chromosomes (BACs). These have been used extensively in the human genome sequencing project (see Cheung et al., Nature, 2001, 409: 953-958) and clones containing specific BACs are available through distributors that can be located through many sources, e.g., NCBI. Each BAC clone from the human genome has been given a reference name that unambiguously identifies it. These names can be used to find a corresponding GenBank sequence and to order copies of the clone from a distributor.

2. Microarrays

Different kinds of biological assays are called microarrays including, but not limited to: DNA microarrays (e.g., cDNA microarrays and oligonucleotide microarrays); protein microarrays; tissue microarrays; transfection or cell microarrays; chemical compound microarrays; and, antibody microarrays. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes by comparing gene expression in disease and normal cells. Microarrays can be fabricated using a variety of technologies, including but not limited to: printing with fine-pointed pins onto glass slides; photolithography using pre-made masks; photolithography using dynamic micromirror devices; ink-jet printing; or, electrochemistry on microelectrode arrays.

Southern and Northern blotting may be used to detect specific DNA or RNA sequences, respectively. In these techniques DNA or RNA is extracted from a sample, fragmented, electrophoretically separated on a matrix gel, and transferred to a membrane filter. The filter bound DNA or RNA is subject to hybridization with a labeled probe complementary to the sequence of interest. Hybridized probe bound to the filter is detected. A variant of the procedure is the reverse Northern blot, in which the substrate nucleic acid that is affixed to the membrane is a collection of isolated DNA fragments and the probe is RNA extracted from a tissue and labeled.

C. Amplification

Chromosomal rearrangements of genomic DNA and chimeric mRNA may be amplified prior to or simultaneous with detection. Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

The polymerase chain reaction (U.S. Pat. Nos. 4,683,195, 4,683, each of which is herein incorporated by reference in its entirety), commonly referred to as PCR, uses multiple cycles of denaturation, annealing of primer pairs to opposite strands, and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA. For other various permutations of PCR see, e.g., U.S. Pat. Nos. 4,683,195, 4,683,202 and 4,800,159; Mullis et al., Meth. Enzymol., 1987, 155: 335; and, Murakawa et al., DNA, 1988, 7: 287.

Transcription mediated amplification (U.S. Pat. Nos. 5,480,784 and 5,399,491, each of which is herein incorporated by reference in its entirety), commonly referred to as TMA, synthesizes multiple copies of a target nucleic acid sequence autocatalytically under conditions of substantially constant temperature, ionic strength, and pH in which multiple RNA copies of the target sequence autocatalytically generate additional copies. See, e.g., U.S. Pat. Nos. 5,399,491 and 5,824,518, each of which is herein incorporated by reference in its entirety. In a variation described in U.S. Pat. No. 7,374,885 (herein incorporated by reference in its entirety), TMA optionally incorporates the use of blocking moieties, terminating moieties, and other modifying moieties to improve TMA process sensitivity and accuracy.

The ligase chain reaction (Weiss, R., Science, 1991, 254: 1292), commonly referred to as LCR, uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product.

Strand displacement amplification (Walker, G. et al., Proc. Natl. Acad. Sci. USA, 1992, 89: 392-396; U.S. Pat. Nos. 5,270,184 and 5,455,166), commonly referred to as SDA, uses cycles of annealing pairs of primer sequences to opposite strands of a target sequence, primer extension in the presence of a dNTPαS to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method (EP Pat. No. 0 684 315).

Other amplification methods include, for example: nucleic acid sequence based amplification (U.S. Pat. No. 5,130,238, herein incorporated by reference in its entirety), commonly referred to as NASBA; one that uses an RNA replicase to amplify the probe molecule itself (Lizardi et al., BioTechnol., 1988, 6: 1197), herein incorporated by reference in its entirety), commonly referred to as Qβ replicase; a transcription based amplification method (Kwoh et al., Proc. Natl. Acad. Sci. USA, 1989, 86:1173); and, self-sustained sequence replication (Guatelli et al., Proc. Natl. Acad. Sci. USA, 1990, 87: 1874). For further discussion of known amplification methods see Persing, David H., “In Vitro Nucleic Acid Amplification Techniques” in Diagnostic Medical Microbiology: Principles and Applications (Persing et al., Eds.), pp. 51-87 (American Society for Microbiology, Washington, D.C. (1993)).

D. Detection Methods

Non-amplified or amplified gene fusion nucleic acids can be detected by any conventional means. For example, the gene fusions can be detected by hybridization with a detectably labeled probe and measurement of the resulting hybrids. Illustrative non-limiting examples of detection methods are described below.

One illustrative detection method, the Hybridization Protection Assay (HPA) involves hybridizing a chemiluminescent oligonucleotide probe (e.g., an acridinium ester-labeled (AE) probe) to the target sequence, selectively hydrolyzing the chemiluminescent label present on unhybridized probe, and measuring the chemiluminescence produced from the remaining probe in a luminometer. See, e.g., U.S. Pat. No. 5,283,174; Nelson et al., Nonisotopic Probing, Blotting, and Sequencing, ch. 17 (Larry J. Kricka ed., 2d ed. 1995).

Another illustrative detection method provides for quantitative evaluation of the amplification process in real-time. Evaluation of an amplification process in “real-time” involves determining the amount of amplicon in the reaction mixture either continuously or periodically during the amplification reaction, and using the determined values to calculate the amount of target sequence initially present in the sample. A variety of methods for determining the amount of initial target sequence present in a sample based on real-time amplification are well known in the art. These include methods disclosed in U.S. Pat. Nos. 6,303,305 and 6,541,205. Another method for determining the quantity of target sequence initially present in a sample, but which is not based on a real-time amplification, is disclosed in U.S. Pat. No. 5,710,029.

Amplification products may be detected in real-time through the use of various self-hybridizing probes, most of which have a stem-loop structure. Such self-hybridizing probes are labeled so that they emit differently detectable signals, depending on whether the probes are in a self-hybridized state or an altered state through hybridization to a target sequence. By way of non-limiting example, “molecular torches” are a type of self-hybridizing probe that includes distinct regions of self-complementarity (referred to as “the target binding domain” and “the target closing domain”) which are connected by a joining region (e.g., non-nucleotide linker) and which hybridize to each other under predetermined hybridization assay conditions. In a preferred embodiment, molecular torches contain single-stranded base regions in the target binding domain that are from 1 to about 20 bases in length and are accessible for hybridization to a target sequence present in an amplification reaction under strand displacement conditions. Under strand displacement conditions, hybridization of the two complementary regions, which may be fully or partially complementary, of the molecular torch is favored, except in the presence of the target sequence, which will bind to the single-stranded region present in the target binding domain and displace all or a portion of the target closing domain. The target binding domain and the target closing domain of a molecular torch include a detectable label or a pair of interacting labels (e.g., luminescent/quencher) positioned so that a different signal is produced when the molecular torch is self-hybridized than when the molecular torch is hybridized to the target sequence, thereby permitting detection of probe:target duplexes in a test sample in the presence of unhybridized molecular torches. Molecular torches and a variety of types of interacting label pairs, including fluorescence resonance energy transfer (FRET) labels, are disclosed in, for example U.S. Pat. Nos. 6,534,274 and 5,776,782.

The interaction between two molecules can also be detected, e.g., using fluorescence energy transfer (FRET) (see, for example, Lakowicz et al., U.S. Pat. No. 5,631,169; Stavrianopoulos et al., U.S. Pat. No. 4,968,103). A fluorophore label is selected such that a first donor molecule's emitted fluorescent energy will be absorbed by a fluorescent label on a second, ‘acceptor’ molecule, which in turn is able to fluoresce due to the absorbed energy.

Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label should be maximal. A FRET binding event can be conveniently measured through standard fluorometric detection means well known in the art (e.g., using a fluorimeter).

Another example of a detection probe having self-complementarity is a “molecular beacon.” Molecular beacons include nucleic acid molecules having a target complementary sequence, an affinity pair (or nucleic acid arms) holding the probe in a closed conformation in the absence of a target sequence present in an amplification reaction, and a label pair that interacts when the probe is in a closed conformation. Hybridization of the target sequence and the target complementary sequence separates the members of the affinity pair, thereby shifting the probe to an open conformation. The shift to the open conformation is detectable due to reduced interaction of the label pair, which may be, for example, a fluorophore and a quencher (e.g., DABCYL and EDANS). Molecular beacons are disclosed, for example, in U.S. Pat. Nos. 5,925,517 and 6,150,097.

Other self-hybridizing probes are well known to those of ordinary skill in the art. By way of non-limiting example, probe binding pairs having interacting labels, such as those disclosed in U.S. Pat. No. 5,928,862 might be adapted for use in meothd of embodiments of the present disclsoure. Probe systems used to detect single nucleotide polymorphisms (SNPs) might also be utilized in the present invention. Additional detection systems include “molecular switches,” as disclosed in U.S. Publ. No. 20050042638. Other probes, such as those comprising intercalating dyes and/or fluorochromes, are also useful for detection of amplification products methods of embodiments of the present disclosure. See, e.g., U.S. Pat. No. 5,814,447.

### VIII. Protein Detection

The gene fusions of the present disclsoure may be detected as truncated or chimeric proteins using a variety of protein techniques known to those of ordinary skill in the art, including but not limited to: protein sequencing and immunoassays. Some of the contemplated techniques are briefly outlined below.

A. Sequencing

Illustrative non-limiting examples of protein sequencing techniques include, but are not limited to, mass spectrometry and Edman degradation.

Mass spectrometry can, in principle, sequence any size protein. A protein is digested by an endoprotease, and the resulting solution is passed through a high pressure liquid chromatography column. At the end of this column, the solution is sprayed out of a narrow nozzle charged to a high positive potential into the mass spectrometer. The charge on the droplets causes them to fragment until only single ions remain. The peptides are then fragmented and the mass-charge ratios of the fragments measured. The mass spectrum is analyzed by computer and often compared against a database of previously sequenced proteins in order to determine the sequences of the fragments. The process is then repeated with a different digestion enzyme, and the overlaps in sequences are used to construct a sequence for the protein.

In the Edman degradation reaction (see, e.g., Edman, Acta Chem. Scand., 1950, 4:283-93), the peptide to be sequenced is adsorbed onto a solid surface (e.g., a glass fiber coated with polybrene). Though there are various well known modifications to this procedure (including automated modifications), one exemplary method involves the use of the Edman reagent, phenylisothiocyanate (PITC), which is added, together with a mildly basic buffer solution of 12% trimethylamine, to an adsorbed peptide, and which reacts with the amine group of the N-terminal amino acid of the adsorbed peptide. The terminal amino acid derivative can then be selectively detached by the addition of anhydrous acid. The derivative isomerizes to give a substituted phenylthiohydantoin, which can be washed off and identified by chromatography, and the cycle can be repeated. The efficiency of each step is about or over 98%, which allows about 50 amino acids to be reliably determined.

B. Immunoassays

Illustrative non-limiting examples of immunoassays include, but are not limited to: immunoprecipitation; Western blot; ELISA; immunohistochemistry; immunocytochemistry; immunochromatography; flow cytometry; and, immuno-PCR. Polyclonal or monoclonal antibodies detectably labeled using various techniques known to those of ordinary skill in the art (e.g., colorimetric, fluorescent, chemiluminescent or radioactive labels) are suitable for use in the immunoassays.

Immunoprecipitation is the technique of precipitating an antigen out of solution using an antibody specific to that antigen. The process can be used to identify proteins or protein complexes present in cell extracts by targeting a specific protein or a protein believed to be in the complex. The complexes are brought out of solution by insoluble antibody-binding proteins isolated initially from bacteria, such as Protein A and Protein G. The antibodies can also be coupled to sepharose beads that can easily be isolated out of solution. After washing, the precipitate can be analyzed using mass spectrometry, Western blotting, or any number of other methods for identifying constituents in the complex.

A Western blot, or immunoblot, is a method to detect protein in a given sample of tissue homogenate or extract. It uses gel electrophoresis to separate denatured proteins by mass. The proteins are then transferred out of the gel and onto a membrane, typically polyvinyldiflroride or nitrocellulose, where they are probed using antibodies specific to the protein of interest. As a result, researchers can examine the amount of protein in a given sample and compare levels between several groups.

An ELISA, short for Enzyme-Linked ImmunoSorbent Assay, is a biochemical technique to detect the presence of an antibody or an antigen in a sample. It utilizes a minimum of two antibodies, one of which is specific to the antigen and the other of which is coupled to an enzyme. The second antibody will cause a chromogenic or fluorogenic substrate to produce a signal. Variations of ELISA include sandwich ELISA, competitive ELISA, and ELISPOT. Because the ELISA can be performed to evaluate either the presence of antigen or the presence of antibody in a sample, it is a useful tool both for determining serum antibody concentrations and also for detecting the presence of antigen.

Immunohistochemistry and immunocytochemistry refer to the process of localizing proteins in a tissue section or cell, respectively, via the principle of antigens in tissue or cells binding to their respective antibodies. Visualization is enabled by tagging the antibody with color producing or fluorescent tags. Typical examples of color tags include, but are not limited to, horseradish peroxidase and alkaline phosphatase. Typical examples of fluorophore tags include, but are not limited to, fluorescein isothiocyanate (FITC) or phycoerythrin (PE).

Flow cytometry is a technique for counting, examining and optionally sorting microscopic particles or cells suspended in a stream of fluid. It allows simultaneous multiparametric analysis of the physical and/or chemical characteristics of single cells flowing through an optical/electronic detection apparatus. A beam of light (e.g., a laser) of a single frequency or color is directed onto a hydrodynamically focused stream of fluid. A number of detectors are aimed at the point where the stream passes through the light beam; one in line with the light beam (Forward Scatter or FSC) and several perpendicular to it (Side Scatter (SSC) and one or more fluorescent detectors). Each suspended particle passing through the beam scatters the light in some way, and fluorescent chemicals in the particle may be excited into emitting light at a lower frequency than the light source. The combination of scattered and fluorescent light is picked up by the detectors, and by analyzing fluctuations in brightness at each detector, one for each fluorescent emission peak, it is possible to deduce various facts about the physical and chemical structure of each individual particle. FSC correlates with the cell volume and SSC correlates with the density or inner complexity of the particle (e.g., shape of the nucleus, the amount and type of cytoplasmic granules or the membrane roughness).

Immuno-polymerase chain reaction (IPCR) utilizes nucleic acid amplification techniques to increase signal generation in antibody-based immunoassays. Because no protein equivalence of PCR exists, that is, proteins cannot be replicated in the same manner that nucleic acid is replicated during PCR, the only way to increase detection sensitivity is by signal amplification. The target proteins are bound to antibodies which are directly or indirectly conjugated to oligonucleotides. Unbound antibodies are washed away and the remaining bound antibodies have their oligonucleotides amplified. Protein detection occurs via detection of amplified oligonucleotides using standard nucleic acid detection methods, including real-time methods.

Methods of antibody generation are a matter of routine skill in the art. See, e.g., U.S. Pat. Nos. 7,923,221, 6,331,415, 4,816,567. For example, antibodies can be prepared by immunizing a suitable mammalian host using, for example, a marker (e.g., PSMA, PSA, a chimeric or truncated protein resulting from an ARG-ETS fusion, etc.) protein, peptide, or fragment, in isolated or immunoconjugated form (Antibodies: A Laboratory Manual, CSH Press, Eds., Harlow, and Lane (1988); Harlow, Antibodies, Cold Spring Harbor Press, NY (1989)). In addition, fusion proteins of markers can also be used, such as a marker-GST-fusion protein. In one embodiment, a GST fusion protein comprising all or most of the amino acid sequence of a marker polypeptide is produced, then used as an immunogen to generate appropriate antibodies. In another embodiment, a marker protein is synthesized and used as an immunogen.

In addition, naked DNA immunization techniques known in the art are used (with or without purified a marker proteins or a marker expressing cells) to generate an immune response to the encoded immunogen. See, e.g, Donnelly et al., Ann. Rev. Immunol., 1997, 15: 617-648.

The amino acid sequence of a marker protein can be analyzed to select specific regions of the protein for generating antibodies. For example, hydrophobicity and hydrophilicity analyses of a marker amino acid sequence are used to identify hydrophilic regions in the structure. Regions of a marker protein that show immunogenic structure, as well as other regions and domains, can readily be identified using various other methods known in the art, such as Chou-Fasman, Garnier-Robson, Kyte-Doolittle, Eisenberg, Karplus-Schultz or Jameson-Wolf analysis. Hydrophilicity profiles can be generated using the method of Hopp & Woods, Proc. Natl. Acad. Sci. U.S.A., 1981, 78: 3824-3828. Hydropathicity profiles can be generated using the method of Kyte & Doolittle, J. Mol. Biol., 1982, 157: 105-132. Percent (%) Accessible Residues profiles can be generated using the method of Janin, Nature, 1979, 277: 491-492. Average Flexibility profiles can be generated using the method of Bhaskaran & Ponnuswamy, Int. J. Pept. Protein Res., 1988, 32: 242-255. Beta-turn profiles can be generated using the method of Deleage & Roux, Protein Engineering, 1987, 1289-294. Thus, each region identified by any of these programs or methods is within the scope of the present invention. Methods for preparing a protein or polypeptide for use as an immunogen are well known in the art. Also well known in the art are methods for preparing immunogenic conjugates of a protein with a carrier, such as BSA, KLH or other carrier protein. In some circumstances, direct conjugation using, for example, carbodiimide reagents are used; in other instances linking reagents such as those supplied by Pierce Chemical Co., Rockford, Ill., are effective.

Administration of a marker immunogen is often conducted by injection over a suitable time period and with use of a suitable adjuvant, as is understood in the art. During the immunization schedule, liters of antibodies can be taken to determine adequacy of antibody formation.

Anti-marker monoclonal antibodies can be produced by various means well known in the art. For example, immortalized cell lines that secrete a desired monoclonal antibody are prepared using the standard hybridoma technology of Kohler and Milstein or modifications that immortalize antibody-producing B cells, as is generally known. Immortalized cell lines that secrete the desired antibodies are screened by immunoassay in which the antigen is a marker protein. When the appropriate immortalized cell culture is identified, the cells can be expanded and antibodies produced either from in vitro cultures or from ascites fluid.

The antibodies or fragments of the invention can also be produced, by recombinant means. Regions that bind specifically to the desired regions of marker protein can also be produced in the context of chimeric or complementarity determining region (CDR) grafted antibodies of multiple species origin. Humanized or human anti-marker antibodies can also be produced, and are preferred for use in therapeutic contexts. Methods for humanizing murine and other non-human antibodies, by substituting one or more of the non-human antibody CDRs for corresponding human antibody sequences, are well known. See, e.g., Jones et al., Nature, 1986, 321: 522-525; Riechmann et al., Nature, 1988, 332: 323-327; Verhoeyen et al., Science, 1988, 239: 1534-1536. See also, Carter et al., Proc. Natl. Acad. Sci. USA, 1993, 89: 4285 and Sims et al., J. Immunol., 1993, 151: 2296.

Methods for producing fully human monoclonal antibodies include phage display and transgenic methods. See, e.g., Vaughan et al., Nature Biotechnology, 1998, 16: 535-539. Fully human anti-marker monoclonal antibodies can be generated using cloning technologies employing large human Ig gene combinatorial libraries (i.e., phage display). See, e.g., Griffiths & Hoogenboom, “Building an in vitro immune system: human antibodies from phage display libraries.” In: PROTEIN ENGINEERING OF ANTIBODY MOLECULES FOR PROPHYLACTIC AND THERAPEUTIC APPLICATIONS IN MAN 45-64 (Clark, M. ed., Nottingham Academic 1993); Burton & Barbas, “Human Antibodies from combinatorial libraries,” id. at 65-82. Fully human anti-marker monoclonal antibodies can also be produced using transgenic mice engineered to contain human immunoglobulin gene loci as described in, for example, PCT Publication No. WO199824893; Jakobovits, Exp. Opin. Invest. Drugs, 1998, 7 (4): 607-614; U.S. Pat. Nos. 6,162,963; 6,150,584; and, 6,114,598. This method avoids the in vitro manipulation required with phage display technology and efficiently produces high affinity authentic human antibodies.

Reactivity of anti-marker antibodies with a marker protein can be established by a number of well known means, including Western blot, immunoprecipitation, ELISA, and FACS analyses using, as appropriate, marker proteins, marker expressing cells or extracts thereof. An anti-marker antibody or fragment thereof can be labeled with a detectable marker or conjugated to a second molecule. Suitable detectable markers include, but are not limited to, a radioisotope, a fluorescent compound, a bioluminescent compound, chemiluminescent compound, a metal chelator or an enzyme. Further, bi-specific antibodies specific for two or more marker epitopes are generated using methods generally known in the art.

### IX. Compositions & Kits

Any of these compositions, alone or in combination with other compositions of the present disclosure, may be provided in the form of a kit. For example, the single labeled probe and pair of amplification oligonucleotides may be provided in a kit for the amplification and detection of gene fusions of the present invention. Kits may further comprise appropriate controls and/or detection reagents. The probe and antibody compositions of the present disclosure may also be provided in the form of a kit or an array.

Compositions for use in the diagnostic methods of the present invention include, but are not limited to, probes, amplification oligonucleotides, and antibodies.

### X. Companion Diagnostics

In some embodiments, the present disclosure provides compositions and methods for determining a treatment course of action in response to a subject's disease marker status. For example, when evaluating an antiandrogen or androgen antagonistic therapeutic it is important to determine the efficacy of the therapeutic in the tested patient population. The present invention may be used not only to evaluate efficacy by way of determining a measurement of prostate cell kill, but also is utilized to identify patient populations that may benefit from one or more specific treatment protocols or therapeutic compounds. For example, where a certain therapeutic compound is known or suspected to be effective in subjects that are afflicted with a cancer that is positive for ARG:ETS gene fusions, or any particular ARG:ETS gene fusion, the present invention is useful to identify these subjects, monitor the effects of treatment with the compound, and determine if the effects are therapeutically beneficial. Determination of whether the effects are therapeutically beneficial may be based on an indication of a positive prostate cell kill. Methods described herein provide an indication of a positive prostate cell kill. In addition, where a certain therapeutic compound is known or suspected to be effective in subjects that test positive for a particular androgen-modulated diagnostic marker, the present invention is useful to identify these subjects and monitor the effects of treatment with the compound. Similarly, where the presence of a particular diagnostic marker in a subjects, which is not an androgen-modulated diagnostic marker, signals that a subjects may benefit from treatment with an antiandrogen composition, the present methods are useful to identify and monitor treatment of these patients.

Conversely, the present methods are useful to identify subjects that are expected to benefit less, or even have a heightened risk of adverse effects if administered a first antiandrogen compound, based on the presence of a particular androgen-modulated diagnostic marker, or other marker. In such circumstances alternative therapy may be medically indicated, for example by administration of a second or other antiandrogen compound. In such circumstances the present invention is also useful to monitor the diagnosis, prognosis, and/or theranosis of these subjects.

### XI. Drug Screening Applications

In some embodiments, the present disclosure provides drug screening assays (e.g., to screen for anticancer drugs). The screening methods of the present disclosure utilize cancer markers identified using the methods of the present invention. For example, in some embodiments, the present disclosure provides methods of screening for compounds that modulate (e.g., decrease) the expression of androgen regulated genes. The compounds or agents may interfere with transcription, by interacting, for example, with the promoter region of the androgen regulated gene. The compounds or agents may also or alternatively interfere with mRNA produced from the fusion (e.g., by RNA interference, antisense technologies, etc.). The compounds or agents may interfere with pathways that are upstream or downstream of the biological activity of the fusion. In some embodiments, candidate compounds are antisense or interfering RNA agents (e.g., oligonucleotides) directed against cancer markers. In other embodiments, candidate compounds are antibodies or small molecules that specifically bind to a cancer marker regulator or expression products of the present disclosure and inhibit its biological function.

Specifically, the present disclosure provides screening methods for identifying modulators, i.e., candidate or test compounds or agents (e.g., proteins, peptides, peptidomimetics, peptoids, small molecules or other drugs) that result in killing cancer cells. Compounds thus identified can be used to attack the cancer cells either directly or indirectly in a therapeutic protocol, to elaborate the biological function of the target gene product, or to identify compounds that disrupt normal target gene interactions.

The test compounds of the present disclosure can be obtained using any of the numerous approaches in combinatorial library methods known in the art, including biological libraries; peptoid libraries (see, e.g., Zuckennann et al., J. Med. Chem., 1994, 37: 2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (see, e.g., Lam, Anticancer Drug Des., 1997, 12:145).

Examples of methods for the synthesis of molecular libraries can be found in the art, for example in: DeWitt et al., Proc. Natl. Acad. Sci. U.S.A., 1993, 90:6909; Erb et al., Proc. Nad. Acad. Sci. USA, 1994, 91:11422; Zuckermann et al., J. Med. Chem., 1994, 37:2678; Cho et al., Science, 1993, 261:1303; Carrell et al., Angew. Chem. Int. Ed. Engl., 1994, 33:2059; Carell et al., Angew. Chem. Int. Ed. Engl., 1994, 33:2061; and Gallop et al., J. Med. Chem., 1994, 37:1233.

Libraries of compounds may be presented in solution (e.g., Houghten, Biotechniques, 1992, 13:412-421), or on beads (Lam, Nature, 1991, 354:82-84), chips (Fodor, Nature, 1993, 364:555-556), bacteria or spores (U.S. Pat. No. 5,223,409; herein incorporated by reference), plasmids (Cull et al., Proc. Nad. Acad. Sci. USA, 1992, 89:18651869) or on phage (Scott and Smith, Science, 1990, 249:386-390; Devlin, Science, 1990, 249:404-406; Cwirla et al., Proc. Natl. Acad. Sci. USA, 1990, 87:6378-6382; Felici, J. Mol. Biol., 1991, 222:301).

### REFERENCES

1. Bruchovsky et al., J. Biol. Chem., 1968, 243, 2012-2021
2. Chen et al., Nat. Med., 2004, 10, 33-39
3. Crawford, et al., J. Urol., 1992, 147: 417A
4. Crawford, et al., N. Engl. J. Med., 1989, 321, 419-424
5. Denis, Prostate, 1994, 5 (Suppl.), 17s-22s
6. Denmeade et al., Nature Rev. Cancer, 2002, 2: 389-396
7. Hall, J. Steroid Biochem. Molec. Biol., 1991, 40, 527-532
8. Huggins et al., Arch. Surg., 1941, 43, 209-212
9. Jemal et al., CA Cancer J. Clin., 2011, 61:69-80
10. McConnell, Urol. Clin. North Am., 1991, 18: 1-13
11. Mohler et al., Clin. Cancer Res., 2004, 10, 440-448
12. Njar et al., Current Pharm. Design, 1999, 5: 163-180
13. Pannek & Partin, Oncology, 1997, 11: 1273-1278

We claim:

1. A method of evaluating the effectiveness of an antiandrogen treatment in a subject, comprising:
(a) contacting a sample obtained from the subject with an androgen modulated diagnostic marker (AMDM) detection reagent and a prostate-specific, androgen independent, diagnostic marker (PSAIDM) detection reagent and detecting, if present, the androgen modulated diagnostic marker and the PSAIDM in the sample;
(b) comparing a pre- and post-antiandrogen treatment level of the AMDM in the subject; and
(c) comparing a pre- and post-antiandrogen treatment level of the PSAIDM in the subject;
wherein detecting a statistically significant pre-/post-antiandrogen treatment decrease in the AMDM, and a statistically significant pre-/post-antiandrogen treatment increase in the PSAIDM in the subject indicates therapeutic effectiveness of the antiandrogen treatment.

2. A method of identifying an antiandrogen compound capable of killing prostate cancer cells, comprising:
(a) contacting a sample obtained from a human with an AMDM detection reagent and a PSAIDM detection reagent and detecting, if present, the AMDM and the PSAIDM in the sample;
(b) comparing a pre- and post-antiandrogen treatment level of the AMDM in the human, wherein the human received treatment with an antiandrogen compound or a suspected antiandrogen compound; and
(c) comparing the pre- and post-antiandrogen treatment levels of the PSAIDM in the human;
wherein detecting a statistically significant pre-/post-antiandrogen treatment decrease in the AMDM, and a statistically significant pre-/post-antiandrogen treatment increase in the PSAIDM in the human identifies an antiandrogen compound capable of killing prostate cancer cells in a human.

3. A method of identifying a human suspected of responding more, or less, favorably to treatment with an antiandrogen compound and monitoring the treatment thereof, comprising:
(a) determining whether a human is suspected of responding more, or less, favorably to treatment with a first antiandrogen compound by examining a sample obtained from the human for the presence, or absence, of a first AMDM, wherein detection of the presence of the first AMDM marker indicates that the human is suspected of responding more, or less, favorably to treatment with the first antiandrogen compound;
(b) for a human identified as suspected of responding more favorably to treatment with the first antiandrogen compound:
(1) examining a sample obtained from the human by contacting the sample with an AMDM detection reagent and a PSAIDM detection reagent and detecting, if present, one or more AMDM and one or more PSAIDM in the sample; and
(2) comparing a pre- and post-treatment level of the one or more AMDM in the human, and comparing a pre- and post-treatment level of the PSAIDM in the human, wherein the human received treatment with the first particular antiandrogen compound; and
(c) for a human identified as suspected of responding less favorably to treatment with the first antiandrogen compound:
(1) examining a sample obtained from the human by contacting the sample with an AMDM detection reagent and a PSAIDM detection reagent and detecting, if present, one or more AMDM and one or more PSAIDM in the sample; and
(2) comparing a pre- and post-treatment level of the one or more AMDM in the human, and comparing a pre- and post-treatment level of the PSAIDM in the human, wherein the human received treatment with a second antiandrogen compound;
wherein detecting a statistically significant pre-/post-antiandrogen treatment decrease in the AMDM, and a statistically significant pre-/post-antiandrogen treatment increase in the PSAIDM in the human identifies a therapeutic benefit of the first or second antiandrogen compound in the respective human that received the first or second antiandrogen compound.

4. A method of evaluating the effectiveness of an antiandrogen treatment in a subject, comprising:
(a) exposing a sample comprising the AMDM to a primer that is complementary to the AMDM and a polymerase under conditions such that an extension product of the primer is generated, and exposing a sample comprising the PSAIDM to a primer that is complementary to the PSAIDM and a polymerase under conditions such that an extension product of the primer is generated;
(b) detecting the extension product of the primer that is complementary to the AMDM, and detecting the extension product of the primer that is complementary to the PSAIDM; and
(c) comparing a pre- and post-antiandrogen treatment level of the AMDM in the subject; and comparing a pre- and post-antiandrogen treatment level of the PSAIDM in the subject,
wherein detecting a statistically significant pre-/post-antiandrogen treatment decrease in the AMDM, and a statistically significant pre-/post-antiandrogen treatment increase in the PSAIDM in the subject indicates therapeutic effectiveness of the antiandrogen treatment.

5. The method of claim 4, wherein the detecting comprises determining a sequence of the extension product of the primer that is complementary to the AMDM, and determining a sequence of the extension product of the primer that is complementary to the PSAIDM.

6. The method of claim 1, wherein detecting a statistically significant pre-/post-treatment decrease in the AMDM, and a statistically significant pre-/post-treatment increase in the PSAIDM in the patient/human indicates that the antiandrogen therapy resulted in prostate cell death.

7. The method of claim 1, wherein the AMDM and the PSAIDM are measured in a blood or a urine sample.

8. The method of claim 1, wherein the AMDM comprises prostate specific antigen (PSA), prostate cancer antigen 3 (PCA3), one or more fusions of an androgen regulated gene with an ETS family member gene, or a combination of two or more of these diagnostic markers.

9. The method of claim 1, wherein AMDM comprises PCA3 and one or more fusions of an androgen regulated gene with an ETS family member gene.

10. The method of claim 1, wherein the PSAIDM comprises PSMA.

11. The method of claim 1, wherein one or more measurements of the AMDM and the PSAIDM are taken before antiandrogen treatment.

12. The method of claim 1, wherein more than one measurement of the AMDM and the PSAIDM are taken after antiandrogen treatment.

13. The method of claim 12, wherein the measurements taken after antiandrogen treatment are periodic measurements over time.

14. The method of claim 13, wherein a kinetic profile of the androgen modulated diagnostic marker and the PSAIDM are generated based on the periodic measurements over time.

15. The method of claim 1, wherein the statistically significant pre-/post-treatment decrease in the AMDM, and the statistically significant pre-/post-treatment increase in the PSAIDM is determined relative to the typical variability range of these markers in cancer patients in the absence of antiandrogen treatment.

16. The method of claim 1, wherein the cancer is prostate cancer.

17. The method of claim 1, wherein the cancer is metastatic prostate cancer.

18. A kit for use in the method of claim 1.
